(19) United States
(12) Patent Application Publication
(10) Pub. No.: US 2020/0178895 A1
(43) Pub. Date: Jun. 11, 2020

(54) WIRELESS, WEARABLE, AND SOFT BIOMETRIC SENSOR

(71) Applicant: BOARD OF REGENTS, THE UNIVERSITY OF TEXAS SYSTEM, Austin, TX (US)

(72) Inventors: Nanshu LU, Austin, TX (US); Hyoyoung JEONG, Austin, TX (US)

(21) Appl. No.: 16/620,637

(22) PCT Filed: Jun. 6, 2018
(86) PCT No.: PCT/US2018/036201

§ 371 Date: Dec. 9, 2019

Related U.S. Application Data
(60) Provisional application No. 62/516,309, filed on Jun. 7, 2017.

(51) Int. Cl. A61B 5/00; A61B 5/01; A61B 5/113; A61B 5/11; A61B 5/0402; A61B 5/0476; A61B 5/0488; A61B 5/0496; A61B 5/053

(52) U.S. Cl. CPC A61B 5/6833; A61B 5/0002; A61B 5/01; A61B 5/113; A61B 5/053; A61B 5/0402; A61B 5/0476; A61B 5/0488; A61B 5/0496; A61B 5/1121

(57) ABSTRACT

By applying a dry and freeform “cut-and-paste” method, an NFC-enabled wireless tattoo-like stretchable biometric sensor can be fabricated within minutes without using any chemicals, inks, or masks/stencils. This sensor is able to wirelessly receive power via a stretchable inductive coil and an NFC chip integrated on the sensor. Data measured by the sensor can be wirelessly transmitted via the same antenna and NFC chip. The sensor is fully stretchable and conformable to human skin and can follows the mechanical deformation of skin without mechanical and electrical failure or delamination. The sensor is imperceptible to wear and can perform high-fidelity sensing for physiological signals. Depending on where the sensor is applied, possible applications include measuring physiological signals such as skin thermography (body temperature), photometry (pulse oximetry, heartbeat), electrograms (ECG, EEG, EMG, EOG), electrical impedance (skin hydration, body fat) and mechanical motion (seismocaridogram, respiratory rate, joint bending).

### CROSS REFERENCE TO RELATED APPLICATION

This application claims priority to and benefit of U.S. provisional patent application Ser. No. 62/516,309 filed Jun. 7, 2017, which is fully incorporated by reference and made a part hereof.

### GOVERNMENT SUPPORT

This invention was made with government support under Grant No. ECCS1509767 awarded by the U.S. National Science Foundation (NSF) and with government support under Grant No. N00014-16-1-2044 awarded by the U.S. Office of Naval Research (ONR). The government has certain rights in the invention.

### FIELD OF THE INVENTION

The present disclosure relates to wearable electronics and, more specifically, to a soft, wearable biometric sensor that is adhered to the skin and powered wirelessly.

### BACKGROUND

Wearable electronics were once anticipated as a solution to address many healthcare challenges. It was hoped that wearable electronics could continuously track clinically-valuable biometrics and provide real-time analytics. The development of wearable electronics for medical/health sensing, however, has faced roadblocks in recent years due to the challenges of balancing functionality, complexity, and power consumption. As a result, the wearable electronics available today are typically limited to sensing low-value biometrics due to their limited sensitivity, comfort, price (e.g., disposability) and complexity (e.g., fabrication complexity). Accordingly, wearable devices have been considered impractical or impossible for many clinical or health applications.

A need, therefore exists for a wirelessly powered epidermal biometric sensor (i.e., E-tattoo or E-tattoo platform) to address these roadblocks. For simplicity, the E-tattoo should be wirelessly powered and should be manufactured using a simple, time-effective, and cost-effective fabrication process instead of a photolithography process. For comfort, all electrical interconnections (including an antenna) should have a shape the provides flexible and stretchable mechanical properties so that the E-tattoo can be laminated on any part of an epidermis, like a temporary transfer tattoo, and fully conform to the microscopic morphology of human skin. Further, the E-tattoo should have a sensitivity and immunity to motion artifacts that is sufficient to measure physiological valuable signals. In addition to body temperature and pulse oximetry, the E-tattoo should also be capable of measuring photometry (pulse oximetry, heartbeat, respiration, blood pressure), electrograms (ECG, EEG, EMG, EOG), and electrical impedance (skin hydration, body fat).

### SUMMARY

Accordingly, in one aspect, the present disclosure embraces a biometric sensor. The biometric sensor includes a flexible, stretchable substrate that can be adhered to an epidermis and that is conformable and bendable with movement of the epidermis. The biometric sensor also includes a circuit disposed on the flexible substrate. The circuit includes an antenna that transmits and receives radio-frequency (RF) signals wirelessly. The circuit also includes a communication module that is electrically connected to the antenna and receives the RE signals from the antenna and converts them into a power. The circuit also includes one or more sensors that are electrically connected to the communication module. The one or more sensors and the communication module are operationally energized by the power to sense biometric signals and transmit the sensed biometric signals wirelessly via the antenna.

In another aspect, the present disclosure embraces a biometric sensing system. The biometric sensing system includes an interrogator device that transmits power signals wirelessly using an interrogator antenna. The biometric sensing system also includes an epidermal sensor that is adhered to an epidermis of a subject. The epidermal sensor includes an epidermal antenna that is inductively coupled with the interrogator antenna to receive the power signals. The power signals operationally energize the epidermal sensor and cause the epidermal sensor to (i) sense biometric signals from the subject, (ii) convert the sensed biometric signals into digitized signals, and (iii) transmit the digitized signals back to the interrogator device.

In another aspect, the present disclosure embraces a method for fabricating a wirelessly powered epidermal biometric sensor. The method includes laminating a metallic (e.g., copper) foil onto a sheet of thermal release tape (TRT). Next, a circuit including an antenna and interconnects are cut into the copper foil. Then, the circuit is transferred onto a sheet of water-soluble tape (WST) that is backed by a polyimide (PI) film by heating the TRT. Electronic components are then soldered to the circuit, and the circuit with the soldered electronic components are transferred to a medical dressing film by wetting the WST. Finally, the circuit is covered with a second medical dressing film so that the circuit is sandwiched between the films of medical dressing.

In exemplary embodiments the interconnects may be comprised of various types of solderable metal, including (but not limited to) copper, copper on PI, tin, tin on PI, gold, gold on PI, nickel, nickel on PI, silver, silver on PI, chromium, and chromium on PI.

The foregoing illustrative summary, as well as other exemplary objectives and/or advantages of the disclosure, and the manner in which the same are accomplished, are further explained within the following detailed description and its accompanying drawings.

Other systems, methods, features, and/or advantages will be or may become apparent to one with skill in the art upon examination of the following drawings and detailed description. It is intended that all such additional systems, methods, features and/or advantages be included within this description and be protected by the accompanying claims.

Those skilled in the art will also appreciate that various adaptations and modifications of the preferred and alternative embodiments described above can be configured without departing from the scope and spirit of the disclosure. Therefore, it is to be understood that, within the scope of the appended claims, the disclosure may be practiced other than as specifically described herein.

### BRIEF DESCRIPTION OF THE DRAWINGS

Figure (FIG.) 1 graphically depicts an exemplary NFC-enabled stretchable E-tattoo according to an embodiment of the present disclosure.

FIG. 2 is a block diagram of a biometric sensing system according to an embodiment of the present disclosure.

FIG. 3 is a flow diagram that graphically illustrates the “cut-and-paste” fabrication method according to an embodiment of the present disclosure.

FIG. 4A graphically depicts an exemplary E-tattoo platform operating under the mechanical deformation of folding.

FIG. 4B graphically depicts an exemplary E-tattoo platform operating under the mechanical deformation of twisting.

FIGS. 4C-4F are images that depict an exemplary E-tattoo platform operating under the mechanical deformation of stretching, wherein FIG. 4C illustrates a 0% stretch, FIG. 4D illustrates a 10% stretch, FIG. 4E illustrates a 15% stretch, and FIG. 4F illustrates a 20% stretch.

### DETAILED DESCRIPTION

The present disclosure embraces biometric sensing using a wirelessly powered stretchable E-tattoo. An embodiment of an E-tattoo (i.e., biometric sensor) 100 that can monitor, record, and transmit real-time information about the wearer's biometric signals is shown in FIG. 1. The E-tattoo 100 can receive power over a wireless link 120 (e.g., near field communication, NFC) with an interrogator device 110 (e.g., a smart phone). The wireless link 120 is typically embodied as a near field communication (NFC) link that operates at about 13.56 megahertz (MHz), though other frequencies and forms of wireless communication are contemplated within this disclosure.

The E-tattoo 100 generally comprises a biometric sensing unit, an energy-harvesting unit, electrical interconnections, and encapsulation (or dielectric) layers. The biometric sensing unit integrates electrodes, a thermistor, different colors of LEDs, one or more photodetectors, operational amplifiers, passive RC filters, analog-front-end (AFE) ICs, and a microcontroller. The energy-harvesting unit is made up of a near-field communication (NFC) IC, an inductively coupled antenna, and impedance matching components. One of the embodiments of the E-tattoo 100 operates without batteries and can be laminated on any part of an epidermis, like a temporary transfer tattoo.

The E-tattoo can mechanically stretch and bend with the skin of a wearer without mechanical and electrical failure or delamination. Thus, the E-tattoo is comfortable to wear and immune to motion artifacts. In an exemplary embodiment, the E-tattoo is fabricated on a flexible, stretchable substrate 102 that has dimension of about 50 millimeters (mm) by 75 mm. As shown in FIG. 1, the circuit and antenna on the flexible, stretchable substrate may include metallic (e.g., copper) traces (i.e., interconnects) 104 that are serpentine in shape to flex with the flexible, stretchable substrate 102 (and skin). In this way, the antenna and circuit are also flexible and stretchable.

The E-tattoo can measure different physiological signals, such as skin thermography (body temperature), photometry (pulse oximetry, heartbeat, respiration, blood pressure), electrograms (ECG, EEG, EMG, EOG), and electrical impedance (skin hydration, body fat). The E-tattoo can be easily applied to different areas to measure different physiological signals.

The E-tattoo platform can be wirelessly powered by a near-field communication (NFC) and/or radio frequency identification (RFID) interrogator (e.g. smartphone) in proximity (e.g., within 8 centimeters) of the E-tattoo.

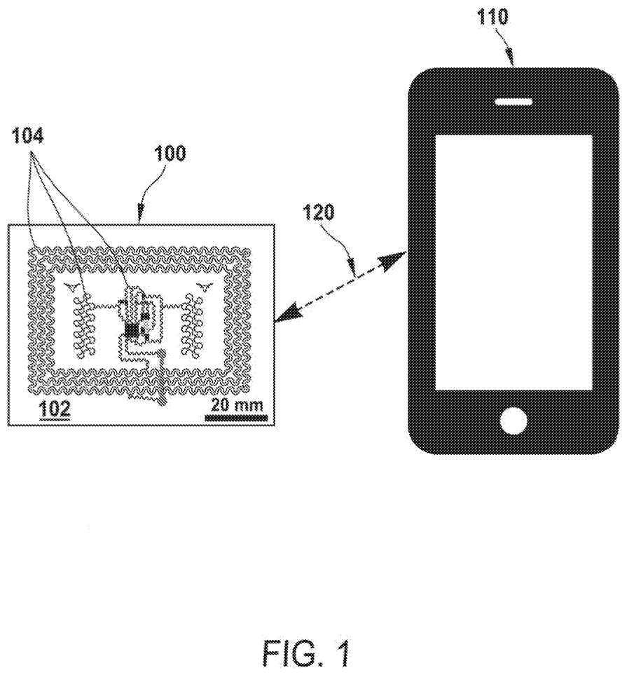

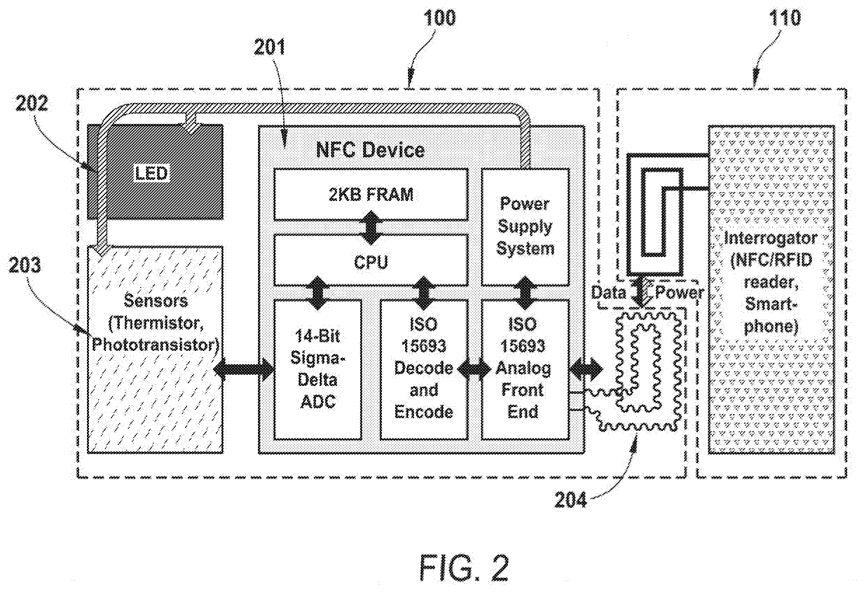

FIG. 2 is a block diagram of a biometric sensing system according to an embodiment of the present disclosure. The E-tattoo 100 includes a communication module 201 (e.g., NFC integrated circuit (IC)). The communication module 201 can store measured data on its own non-volatile FRAM, but also wirelessly transmits the data to the interrogator 110 via an antenna 204. Data may be transferred in real-time or on a periodic basis. In addition to the communication module 201, the E-tattoo may also include active components 202 such as a one or more light emitting diodes (LEDs) and/or sensors 203, such as a thermistor, and photodetector. All electrical interconnections between components including an inductively coupled antenna 204 for transmitting power and data with the interrogator device 110 are designed in such a way that it can acquire flexible and stretchable mechanical properties by applying a serpentine shape. The flexibility facilitates lamination to any part of an epidermis, which yields low electrode-skin interface impedance and enables high signal-to-noise ratio (SNR) measurements.

In one of the embodiments, the NFC IC includes an analog-to-digital converter that digitizes signals from the sensors. The NFC may also include an encoder that encodes the digitized signals into the NFC communication protocol and a transmitter that transmits the encoded signals as RF signals to an interrogator device (e.g., smartphone or RFID reader).

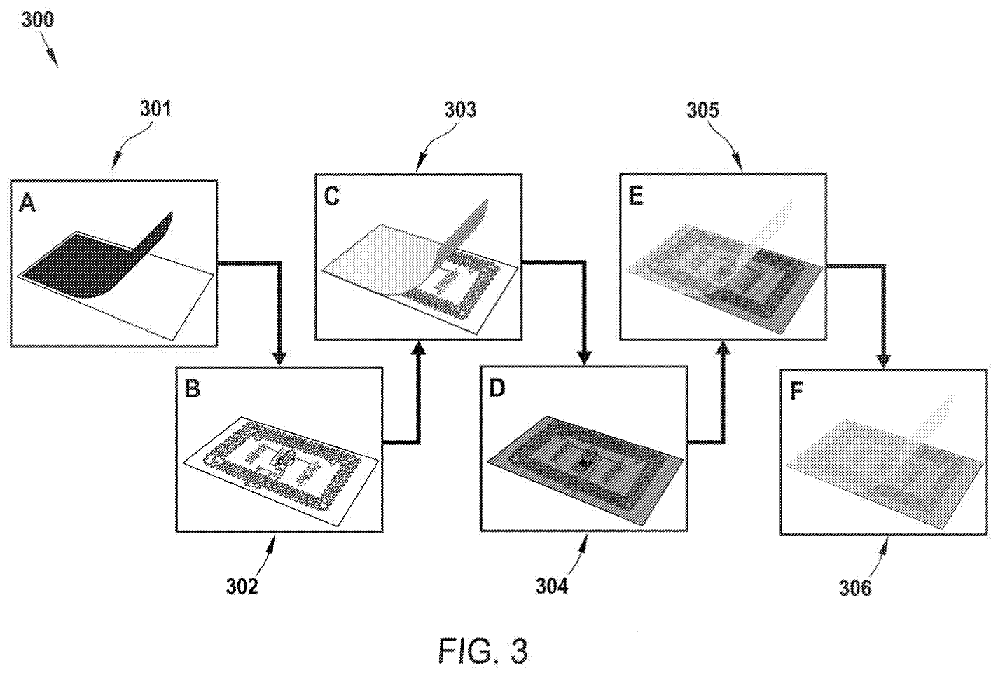

A time/cost effective “cut-and-paste” fabrication method can be used to integrate stretchable metal circuitry and connect electronic components on a flexible, stretchable substrate (e.g., adhesive patch). FIG. 3 graphically illustrates a flow diagram of the “cut-and-paste” fabrication method according to an embodiment of the present disclosure.

As shown in FIG. 3, the E-tattoo considerably differs from present technology in terms of the fabrication method. The fabrication process is more time/cost effective than existing fabrication processes, such as photolithography, metal deposition, dry/wet etching, and transfer printing. In one aspect, a benchtop programmable cutting machine is used to cut (i.e., carve) the circuit patterns forming the E-tattoo.

The method for fabricating a wirelessly powered epidermal biometric sensor 300 comprises laminating 301 a sheet of copper foil onto a sheet of thermal release tape (TRT). The copper foil is cut 302 into a circuit that includes an antenna and interconnects, each having a serpentine shape for flexibility. The TRT is heated so that the circuit can be transferred 303 onto a sheet of water-soluble tape (WST), which is backed by a polyimide film (e.g., KAPTON™ tape). The polyimide film provides a stable support for the circuit so that components may be soldered to the circuit. Accordingly, the process includes soldering 304 components to the circuit. The circuit with the soldered electronic components is then transferred 305 to a medical dressing film (e.g., TEGADERM™) by wetting the WST. The circuit is covered 306 by a second medical dressing film so that the circuit and components) is sandwiched between the films. This electrically insulates the circuit from the skin.

A benchtop programmable cutter plotter used in the cutting 302 operation allows for patterns or shapes to be cut out simply on a thin sheet of polymer, metal-coated polymer, or even atomic sheets such as graphene. Moreover, the “cut-and-paste” method is a fab-less process, making it more cost- and time-effective than a cleanroom process that involves photolithography patterning and chemical etching.

In an exemplary fabrication, an 18-μm-thick copper (Cu) foil is laminated on thermal release tape (TRT). A mechanical cutter plotter cuts the designed circuit pattern onto the Cu foil within a period of minutes. After removing excess Cu foil, the Cu circuit is transferred onto a water-soluble tape (WST) backed by a KAPTON™ tape, which serves as a stable support for soldering. Solder paste is applied to attach the NFC chip and other discrete electrical components on the Cu circuit. Dissolving the WST with wetter droplets, the whole circuit can be transferred to the soft stretchable target substrate, TEGADERM™ (47 μm thick). Finally, another TEGADERM™ is applied from the other side as an encapsulation layer to isolate the circuit from directly touching the skin.

In terms of efficiency, the freeform “cut-and-paste” method is favored over conventional microfabrication methods involving photolithography, metal depositon, and chemical etching because the E-tattoo can be fabricated in ambient environment, within 30 minutes, and using only a simple cutting machine.

Other wearable devices that have attempted to bring wireless powered electronics to temporary tattoo form factor are only capable of measuring basic physiological signals such as body temperature and pulse oximetry. In addition to these signals, the E-tattoo platform can also sense photometric signals (e.g., for pulse oximetry, heartbeat, respiration, blood pressure), electrograms (e.g., for ECG, EEG, EMG, EOG), and electrical impedance (e.g., for skin hydration, body fat).

The E-tattoo platform solves a variety of challenges that exist with current technology. For example, one problem lies in the high-power requirements for wireless communication. To solve this challenge, embodiments of the disclosed E-tattoo obtains operating power by harvesting energy from the electromagnetic field of an external device (e.g., interrogator device) using an inductive loop antenna and conditioned using a Near Field Communication (NFC) integrated circuit (IC). The inductive coupling typically requires the E-tattoo and the interrogator device to be placed proximate (e.g., within 8 centimeters) to one another so that the antenna (e.g., loop) of the E-tattoo is inductively coupled to the antenna of the interrogator device.

The E-tattoo platform also possesses advantages over current technologies. The E-tattoo platform can offer a variety of capability for mechanical, electrical, and efficient aspects. The E-tattoo offers the mechanical advantages of flexibility and stretchability. The flexibility is due, in part, to the mechanics of serpentines. In an exemplary embodiment, a double-stranded serpentine can be used for the antenna coil and interconnections. The double stranded serpentine saves space while maintaining stretchability and compliance. As serpentines do not add much stiffness, the modulus of the E-tattoo will be dominated by that of TEGADERM™ (e.g., 7.4 MPa), which is close to that of human skin (e.g., 0.32-4 MPa). In exemplary embodiments, the flexible, stretchable substrate has a modulus (e.g., Young's modulus) in the range of 0.1 to 10 Mega-Pascals (MPa) to match the flexibility of human skin. The E-tattoo may therefore be laminated on an epidermis like an imperceptible secondary skin without constraining the natural skin deformation.

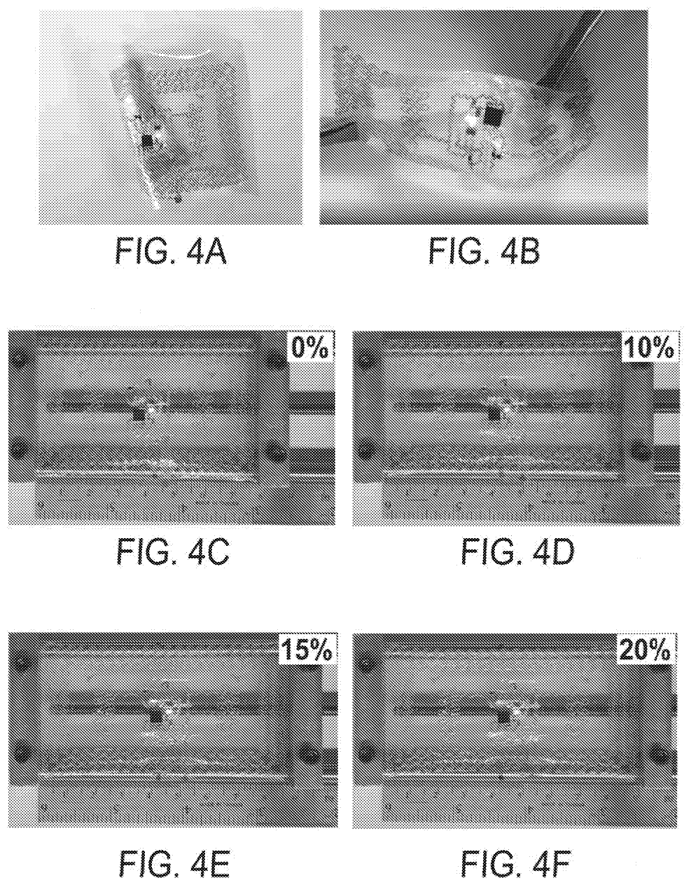

FIGS. 4A-4F are images that illustrate the results of a variety of tests of the flexibility and stretchability of an exemplary E-Tattoo during operation.

FIG. 4A graphically depicts an exemplary E-tattoo platform operating under the mechanical deformation of folding.

FIG. 4B graphically depicts an exemplary E-tattoo platform operating under the mechanical deformation of twisting.

FIGS. 4C-4F graphically depict an exemplary E-tattoo platform operating under the mechanical deformation stretching, wherein FIG. 4C illustrates a 0% stretch, FIG. 4D illustrates a 10% stretch, FIG. 4E illustrates a 15% stretch, and FIG. 4F illusrates a 20% stretch.

The tests show that even after fully wrapping the E-tattoo around less than 10 mm radius of curvature or twist, or stretching it up to 20%, the LED remained on, indicating that the antenna and the interconnections are insensitive to mechanical deformation.

The E-tattoo facilitates the measurements of physiological signals on any skin surface on a human or animal body. Embodiments of the E-tattoo platform may be used in a range of applications. The E-tattoo can be applied in many aspects such as physiological signal monitoring for clinical/biomedical applications (e.g., healthcare wearable devices), fitness/entertainment products (e.g., activity tracker), and/or for animal medicine and health tracking. In particular, the E-tattoo platform can be applied to any part of the body or organ that needs to be monitored for an amount of dosage or exposure (e.g., UV light, radiation, stress, hypo/hyperthermia). The E-tattoo platform can monitor variations in physiological signals (e.g., heartbeat, respiration, body temperature, skin impedance, sweat level, ECG, EEG, EMG, and EOG).

A few exemplary and non-limiting applications of the E-tattoo platform are as follows:

1. Body temperature can be measured by integrating circuit with a thermistor. Output from the thermistor is voltage, and temperature is calculated according to the output voltage.

2. Skin hydration/Body fat can be measured by integrating circuit with an operational amplifier. Output from the amplifier is voltage, and skin conductance is calculated according to the output voltage. Skin conductance be calibrated with a skin hydration/body fat.

3. Pulse oximetry/Heartbeat/respiration can be measured by integrating circuit with green (or IR) LED, red LED, one or more photodetectors, amplifier for amplifying signal from the photodetector, and passive (or active) low/high/band pass filter. Output from the amplifier is voltage, and a peripheral capillary oxygen saturation (SpO2) is calculated according to the output signal difference between green (or IR) LED and red LED. In addition, heartbeat and respiration can be acquired using the measured pulse oximetry signal.

4. Electrograms (ECG, EEG, EMG, and ECG) can be measured by AFE ICs, integrating circuit with instrumentational amplifier, operational amplifier, and low/high/band pass filter. Output from the amplifier is voltage, and ECG/EEG/EMG/EOG is measured according to the output voltage. Blood pressure (BP) can be estimated by the output signals of ECG and a photodetector.

In the specification and/or figures, the use of the term “and/or” includes any and all combinations of one or more of the associated listed items. The figures are schematic representations and so are not necessarily drawn to scale. Unless otherwise noted, specific terms have been used in a generic and descriptive sense and not for purposes of limitation.

While several embodiments have been provided in the present disclosure, it should be understood that the disclosed systems and methods may be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted or not implemented.

Also, techniques, systems, subsystems, and methods described and illustrated in the various embodiments as discrete or separate may be combined or integrated with other systems, modules, techniques, or methods without departing from the scope of the present disclosure. Other items shown or discussed as directly coupled or communicating with each other may be indirectly coupled or communicating through some interface, device, or intermediate component, whether electrically, mechanically, or otherwise. Other examples of changes, substitutions, and alterations are ascertainable by one skilled in the art and could be made without departing from the spirit and scope disclosed herein.

1. A biometric sensor, comprising
a flexible, stretchable substrate that can be adhered to an epidermis, the flexible, stretchable substrate conformable and bendable with movement of the epidermis;
a circuit disposed on the flexible, stretchable substrate, the circuit comprising:
an antenna that transmits and receives radio-frequency (RF) signals wirelessly;
a communication module electrically connected to the antenna that converts the received RF signals into a power,
one or more sensors electrically connected to the communication module, wherein the one or more sensors and the communication module are operationally energized by the power to sense biometric signals and transmit the sensed biometric signals wirelessly via the antenna.

2. The biometric sensor according to claim 1, wherein the flexible, stretchable substrate has a modulus in the range of 0.1 to 10 Mega-Pascals (MPa) to match the flexibility of human skin.

3. The biometric sensor according to claim 1, wherein the flexible, stretchable substrate has dimensions of about 50 millimeters by 75 millimeters.

4. The biometric sensor according to claim 1, wherein the circuit comprises metallic interconnects that are serpentine shaped to flex with the flexible, stretchable substrate.

5. The biometric sensor according to claim 1, wherein the antenna is a loop formed from a serpentine trace.

6. The biometric sensor according to claim 5, wherein the antenna receives the RF signals wirelessly through inductive coupling with another device.

7. The biometric sensor according to claims 5, wherein the antenna comprises a capacitor to tune the resonance frequency of the antenna.

8. The biometric sensor according to claim 7, wherein the resonant frequency is about 13.56 megahertz (MHz).

9. The biometric sensor according to claim 1, wherein that communication module is an integrated circuit chip for near field communication (NFC).

10. The biometric sensor according to claim 9, wherein the integrated circuit chip for NFC comprises:
an analog to digital converter that digitizes signals from the one or more sensors; and
an encoder that encodes the digitized signals into a communication protocol; and
a transmitter that transmits the encoded signals as RF signals to an interrogator device.

11. The biometric sensor according to claim 1, wherein the one or more sensors comprises a thermistor.

12. The biometric sensor according to claim 1, wherein the one or more sensors comprise one or more photodetectors.

13. The biometric sensor according to claim 1, wherein the circuit further comprises one or more LEDs.

14. A biometric sensing system, comprising:
an interrogator device transmitting power signals wirelessly using an interrogator antenna; and
an epidermal sensor adhered to an epidermis of a subject, the epidermal sensor comprising an epidermal antenna that is inductively coupled with the interrogator antenna to receive the power signals, wherein the epidermal sensor is operationally energized by the received power signals to:
sense biometric signals from the subject,
convert the sensed biometric signals into digitized signals, and
transmit the digitized signals back to the interrogator device.

15. The biometric sensing system according to claim 14, wherein the interrogator device is a smart phone.

16. The biometric sensing system according to claim 14, wherein the power signals and digitized signals conform to a near field communication (NFC) protocol.

17. The biometric sensing system according to 16 claim 14, wherein the interrogator device and the epidermal sensor are inductively coupled within a range of 8 centimeters.

18. The biometric sensing system according to claim 14, wherein the epidermal sensor conforms, bends, and stretches with the epidermis without loss of operation or detachment from the epidermis.

19. The biometric sensing system according to claim 14, wherein the biometric parameters correspond to electrophysiological, mechanical, thermal, optical, or electrochemical measurements.

20. A method for fabricating a wirelessly powered epidermal biometric sensor, the method comprising:
laminating a metal foil onto a sheet of thermal release tape (TRT);
cutting the metal foil into a circuit comprising an antenna and interconnects;
transferring the circuit onto a sheet of water soluble tape (WST) that is backed by a polyimide film by heating the TRT;
soldering electronic components to the circuit;
transferring the circuit with soldered electronic components to a medical dressing film by wetting the WST;
covering the circuit with a second medical dressing film so that the circuit is sandwiched between the films of medical dressing.
